(19) United States
(12) Patent Application Publication
(10) Pub. No.: US 2006/0280817 A1
(43) Pub. Date: Dec. 14, 2006

(54) Pharmaceutical composition useful for the treatment of hepatocellular carcinoma

(76) Inventors: Ajit Kumar Saxena, Jammu (IN); Bishan Datt Gupta, Jammu (IN); Bal Krishan Kapahi, Jammu (IN); Shanmugavel Muthiah, Jammu (IN); Dilip Manikrao Mondhe, Jammu (IN); Baleshwar, Jammu (IN); Ghulam Nabi Qazi, Jammu (IN); Vijay Kumar, New Delhi (IN); Ganeshan Mathan, New Delhi (IN)

Correspondence Address: REED SMITH LLP, 2500 ONE LIBERTY PLACE, 1650 MARKET STREET, PHILADELPHIA, PA, 19103, US

(21) Appl. No.: 11/440,790

(22) Filed: May 25, 2006

(30) Foreign Application Priority Data
May 26, 2005 (IN) 1356/DEL/2005

(51) Int. Cl. A61K 36/48

(52) U.S. Cl. 424/757

(57) ABSTRACT

The present invention relates to anticancer activity against hepatocellular carcinoma of an extract and fraction isolated from flowers of Butea monosperma. Particularly, this invention relates to anticancer activity against hepatocellular carcinoma of a composition containing markered flavonoid glycosides such as butrin and isobutrin in the range of 2 to 9% by weight, isolated from the flowers of Butea monosperma by extracting the flowers with polar solvent like ethanol, methanol, aqueous ethanol or water, removing fatty non-polar constituents by triturating the extract with solvents such as ethylene chloride, methylene chloride, chloroform or ethyl acetate, suspending the residue in water, extracting with n-butanol and freeze drying the aqueous part.

This application claims the right of priority under 35 U.S.C. §119(a)-(d) to Indian Patent Application No. 1356/DEL/2005, filed May 26, 2005 and the text of application 1356/DEL/2005 is hereby incorporated by reference in its entirety.

### FIELD OF THE INVENTION

The present invention relates to a pharmaceutical composition useful for the treatment of hepatocellular carcinoma. More particularly, it relates to a method of treating hepatocellular carcinoma in a subject.

The present invention also relates to the use of the extract or its active fraction obtained from any plant parts of Butea monosperma in the treatment of hepatocellular carcinoma.

Further, it also relates to a process for isolating the bioactive fraction comprising of butrin and/or isobutrin from any plant parts of Butea monosperma.

More particularly, it relates to the use of said bioactive fraction and butrin and isobutrin in the treatment of hepatocellular carcinoma.

### BACKGROUND OF THE INVENTION

Butea monosperma (Lam) (family: Fabaceae) is a medium sized tree found in greater parts of India and is reported to have numerous uses in the indigenous system of medicine in India. Various medicinal properties are ascribed to flowers, leaves, bark and roots of this plant. The leaves are astringent, tonic, diuretic and aphrodisiac. They are used to cure boils. The bark is reported to possess astringent, bitter, pungent, alterative, aphrodisiac and antihelmintic properties. The roots are useful in elephantiasis and in curing night blindness. Flowers are reported to possess astringent, depurative, aphrodisiac and tonic properties (Chopra, R. N., Nayar, S. L. and Chopra, I. C., Glossary of Indian Medicinal Plants, CSIR, New Delhi, 1956, p. 42; Wealth of India: Raw Material, CSIR, New Delhi, (1988) Vol. 2B, p. 341-46). The petroleum ether and ethyl acetate extracts of the stem bark have shown anti-fungal activity. (−)-Medicarpin has been identified as active principle (Ratnayake Bandara, B. M., Savitri Kumar, N. and Swama Samaranayake, K. M., Journal of Ethanopharmacology 25(1), 735 (1989)). Hot alcoholic extract of the seeds showed significant anti-implantation and antiovulatory activities in rats and rabbits respectively. It also showed abortive effect in mice (Choudhury, R. R and Khanna, U., Indian Journal of Medical Research, 56(10) 1575, (1968)). Butin, isolated from the seeds of Butea monosperina, has been reported to possess anti-implantation activity in rats (Bhargava, S. K., Journal of Ethanopharmacology 18, 95-101, (1986)). A triterpene isolated from the flowers has been reported as active principle for anticonvulsive activity in laboratory animals (Kasture, V. S., Kasture, S. B. and Chopde, C. T., Pharmacol. Biochem. Behav. 72, 965-972 (2002)). The methanol extract of seeds, tested in vitro, showed significant anthelmintic activity (Prashanth, D. Asha, M. K., Amit, A. and Padmaja, R. Fitoterapia 72, 421-422 (2001)). An “Ayurvedic Rasayana” (herbal medicine) containing Butea monosperma as one of the constituents has been reported for the management of giardiasis perhaps by immunomodulation as the “Rasayana” had no killing effect on the parasite in vitro (Agarwal, A. K., Singh, M., Gupta, N., Saxena, R., Puri, A., Verma, A. K., Saxena R. P., Dubey, C. B., Saxena, K. C. Journal of Ethanopharmacology 44, 143-146 (1994)). Isobutrin and butrin have been identified as the antihepatotoxic principles from flowers of Butea monosperma (Wagner, H., Geyer, B., Fiebig, M., Kiso, Y. and Hikino, H. Planta Medica 77-79 (1986)). Butea monosperma flowers have been reported to possess antistress activity (Bhatwadekar, A. D., Chintawar, S. D., Logade, N. A., Somani, R. S., Kasture, V. S. and Kasture, S. B. Indian Journal of Pharmacology, 31, 153-155 (1999)). To the best of our knowledge, so far, the anticancer activity of any of the plant part or its isolate/constituent has not been reported.

A large number of flavonoids viz. butein, butin, butrin, isobutrin, palasitrin, coreopsin, isocoreopsin, sulphuretin, monospernoside and prunetin have been isolated from the flowers of this plant (Gupta, S. R., Ravindranath, B. and Seshadri, T. R., Phytochemisrty 9, 2231-35 (1970); Puri, B. and Seshadri, T. R. J. Sci. Ind. Res. (India) 12B, 462 (1953); Lal, J. B. and Dutt, S., J. Ind. Chem. Soc., 12, 262 (1935)). Several nitrogenous constituents have also been reported which include palasonin (Raj, R. K. and Karup, P. A., Ind. J. Chem. 5, 86-87 (1967)), monospermin (Mehta, B. K. and Bokadia, M. M., Chem. & Ind. 3, 98 (1981)), allophanic acid derivatives (Porwal, M., Sharma, S. and Mehta, B. K., Ind. J. Chem. 27B, 281-82 (1988)) and palasimide (Guha, P. K., Poi, R. and Bhattacharya, A. Phytochemistry 29, 2017 (1990). Seeds have also been reported to contain α-amyrin, β-sitosterol, β-sitosterol glucoside (Chandra, S., Lal, J. and Sabir, M, Ind. J. Pharmacy 35, 79-80, 1977) and hexeicosanoic acid δ-lactone (Bishnoi, P. and Gupta, P. C. Planta Medica 35, 286-88, (1979)). Palasonin, isolated from seeds showed anthelmintic activity (Kaleysa Raj, R. and Karup, P. A. Ind. Jour. Med. Res. 56, 12, (1968)). From the stems, isolation of two new compounds 3α-hydroxyeuph-25-ene and 2,14-dihydroxy-11,12-dimethyl-8-oxo-octadec-11-enylcyclohexane has been reported (Mishra, M., Shukla, Y. N. and Kumar, S., Phytochemistry 54(8), 835-38, (2000)). From the resin fraction of the seed—lac, isolation of four acid esters designated as jalaric ester I, jalaric ester II, laccijalaric ester I and laccijalaric ester II has been reported (Singh, A. N., Upadhye, V., Mhaskar, V. V. and Dev. S. Tetrahedron, 30, 867-74, (1974)).

The plant is well known for treatment of liver disorders in ISM. The active compounds (butrin and isobutrin) from flowers have been reported for hepatoprotective activity. In a recent research paper entitled “Butea monosperma and chemomodulation: Protective role against thioacetamide—mediated hepatic alternations in Wistar rats by A. Sehrawat, T H Khan, L. Prasad and S. Sultana (Phytomedicine 13. 157-163, 2006) the hepatoprotective action of the plant extract having these compounds has been studied against thioactamide induced hepatotoxicity. Thioactamide is a hazardous, toxic and cacrcinogenic. In the same paper two more parameters i.e. DOC and H3 thymidine incorporation has been studied to demonstrate that in may inhibit tumor formation by inhibiting these two parameters. There is no indication regarding direct anticancer effect of Butea extract. Even the development of cancer in control animals has not been demonstrated and no parameter shows protective action on cancer at the most it may be considered as chemopreventive/anticarcinogenic action. The authors themselves have concluded “Overall results indicate that the methanolic extract of B. Monosperma possess hepatoprotective effect and also it might suppress the promotion stage via inhibition of oxidative stress and polyamine biosynthetic pathway”

### OBJECTS OF THE INVENTION

The main object of the present invention is to provide a pharmaceutical composition useful for the treatment of hepatocellular carcinoma.

Another object of the present invention is to provide a method of treating hepatocellular carcinoma in a subject.

Further, another object of the present invention is to provide a process for isolating the bioactive fraction comprising of butrin and/or isobutrin from any plant parts of Butea monosperma.

Yet another object of the present invention is to provide the use of the extract or its bioactive fraction obtained from any plant parts of the Butea monosperma in the treatment of hepatocellular carcinoma.

Still another object of the present invention is to provide the use of the butrin and isobutrin in the treatment of hepatocellular carcinoma.

### SUMMARY OF THE INVENTION

The present invention deals with a pharmaceutical composition useful for the treatment of hepatocellular carcinoma in a subject wherein the said composition comprising the therapeutically effective amount of an extract or its active fraction obtained from any plant parts of Butea monosperma or therapeutically effective amount or compound butrin and/or isobutrin or its derivatives or analogues or pharmaceutically acceptable salt thereof optionally along with one or more pharmaceutically acceptable carriers. Further, it also relates to a method of treating hepatocellular carcinoma in a subject and a process for isolating the bioactive fraction comprising of butrin and/or isobutrin from any plant parts of Butea monosperma and the use thereof in the treatment of hepatocellular carcinoma.

### BRIEF DESCRIPTION OF THE FIGURES

FIG. 1 represents the general structure of compounds Isobutrin and butrin. Isobutrin (1): m.p. 187-89°; M⁺ 596; ¹H NMR (200 Hz, DMSO-d6) showed signals at δ 6.63 (2H, m, H-3′, H-5′), 6.90 (1H, d, J=8 Hz, H-5), 7.46 (1H,d, J=8 Hz, H-6), 7.72 (3H, m, H-2, H-α, H-β), 8.23 (1H,d, J=8 Hz, H-6′); IR (KBr) ν (cm⁻¹): 3386, 2981, 1633, 1572, 1518, 1421, 1363, 1284, 1219, 1124, 1072, 804

Butrin (2); m.p. 189-90°; M⁺ 596; ¹H NMR (200 MHz, DMSO-d6) showed signals at δ3.18 (2H, m, H-3), 5.45 (1H, dd, J=4, 12 Hz, H-2), 6.68 (1H, d, J=8 Hz, H-8), 6.72 (1H,d, J=8 Hz, H-6), 6.80 (1H,d, J=8 Hz, H-5′), 7.05 (1H,d, J=8 Hz, H-6′), 7.30 (1H,s, H-2′), 7.73 (1H,d, J=8 Hz, H-5) IR (KBr) ν (cm⁻¹): 3362, 2925, 1667, 1613, 1574, 1523, 1443, 1281, 1085, 860, 804.

FIG. 2 represents liver histology of x-myc mice (control, no treatment), A. 12 weeks and B. 20 weeks (All 100×). The liver of control animals showed a typical mitosis, dyslasia and loss of normal hepatic architecture. The malignant hepatocyte cords showed large pleiomorphic nuclei with multinucleation and macronucleoli.

FIG. 3 represents liver histology of x-myc micevtreated with Butea monosperma flowers aqueous extract, A. 12 weeks and B. 20 weeks (All 100×).

FIG. 4 represents liver histology of x-myc mice treated with Butea monosperma flowers fraction, A. 12 weeks and B. 20 weeks (All 100×) where the liver appeared to be normal both at 12 and 20 weeks post-treatment.

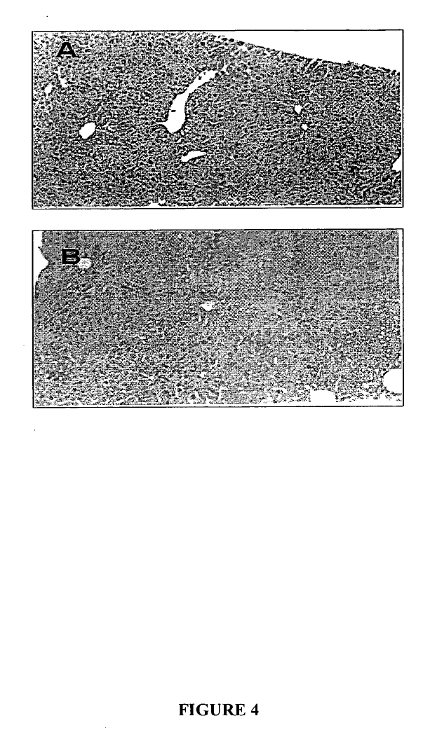

FIG. 5 is a flowchart for isolation of active fraction from Butea monosperma.

### DETAILED DESCRIPTION OF THE INVENTION

Accordingly, the present invention provides a pharmaceutical composition useful for the treatment of hepatocellular carcinoma wherein the said composition comprising the therapeutically effective amount of an extract and/or its active fraction obtained from any plant parts of Butea monosperma or therapeutically effective amount or compound butrin and/or isobutrin or its derivatives or analogues or pharmaceutically acceptable salt thereof optionally along with one or more pharmaceutically acceptable carriers.

In an embodiment of the present invention, the said composition comprising the therapeutically effective amount of an extract and/or its active fraction obtained from any plant parts of Butea monosperma optionally along with one or more pharmaceutically acceptable carriers.

In another embodiment of the present invention, the dosage of the said composition is administered at a unit dose of at least 0.5 g/kg body weight.

Further, in another embodiment of the present invention, the said composition comprising the therapeutically effective amount of compound butrin and/or iso butrin or its derivatives or analogues or pharmaceutically acceptable salt thereof optionally along with one or more pharmaceutically acceptable carriers.

In yet another embodiment of the present invention, the dosage of the said composition is administered at a unit dose of less than 0.5 g/kg body weight.

In still another embodiment of the present invention, the dosage of the said composition is administered in soluble form preferably in suspension form.

In still another embodiment of the present invention, the carrier used is selected from the group consisting of saline, gum acacia, carboxy methyl cellulose or any other known pharmaceutically acceptable carrier.

In still another embodiment of the present invention, the said composition is used systemically, orally or by any clinical, medically accepted methods.

In still another embodiment of the present invention, the administration route is selected from the group comprising of intraperitoneal, intravenous, intramuscular, oral etc.

In still another embodiment of the present invention, the said composition is used for both preventive and curative purpose.

Further, the present invention also provides a method of treating hepatocellular carcinoma in a subject, wherein the said method comprising the step of administering to the subject a pharmaceutical composition comprising the therapeutically effective amount of an extract and/or its active fraction obtained from any plant parts of Butea monosperma or therapeutically effective amount of compound butrin and/or isobutrin or its derivatives or analogues or pharmaceutically acceptable salt thereof optionally along with one or more pharmaceutically acceptable carriers.

In an embodiment of the present invention, the subject is selected from the group consisting of humans and mammals, preferably humans.

In an embodiment of the present invention, the said method comprising the step of administering to the subject a pharmaceutical composition comprising the therapeutically effective amount of an extract and/or its active fraction obtained from any plant parts of Butea monosperma optionally along with one or more pharmaceutically acceptable carriers.

In another embodiment of the present invention, the dosage of the said composition administered is at a unit dose of at least 0.5 g /kg body weight.

Further, in another embodiment of the present invention, the said method comprising the step of administering to the subject a pharmaceutical composition comprising the therapeutically effective amount of compound butrin and/or isobutrin or its derivatives or analogues or pharmaceutically acceptable salt thereof optionally along with one or more pharmaceutically acceptable carriers.

In yet an embodiment of the present invention, the dosage of the said formulation administered is at a unit dose of less than 0.5 g/kg body weight.

In still an embodiment of the present invention, the dosage of the said composition is administered in soluble form preferably in suspension form.

In still an embodiment of the present invention, the carrier used is selected from the group consisting of saline, gum acacia, carboxy methyl cellulose or any other known pharmaceutically acceptable carrier.

In still another embodiment of the present invention, the said composition is used systemically, orally or by any clinical, medically accepted methods.

In still an embodiment of the present invention, the administration route is selected from the group consisting of intraperitoneal, intravenous, intramuscular, oral etc.

The present invention also provides the use of the extract and bioactive fraction obtained from Butea monosperma in the treatment of hepatocellular carcinoma.

In an embodiment of the present invention, the use of the compound butrin and isobutrin is in the treatment of the hepatocellular carcinoma.

Further, the present invention provides a process for isolating the bioactive fraction comprising of butrin and/or isobutrin from any plant parts of Butea monosperma, wherein the said process comprising:
a) powdering the plant material;
b) extracting the powder obtained from step (a) by percolation using solvents selected from the group comprising of ethanol, methanol, water, individually or in combination thereof to obtain extract;
c) concentrating the extract obtained from step (b) under reduced pressure at <50° C.;
d) titrating the extract obtained from step (c) with solvents selected from the group comprising of ethylene chloride, methylene chloride, chloroform &/or ethyl acetate to get residue;
e) partitioning the residue obtained from step (d) between aqueous phase and organic phase;
f) drying the aqueous part obtained from step (e) to get desired active fraction by known methods.

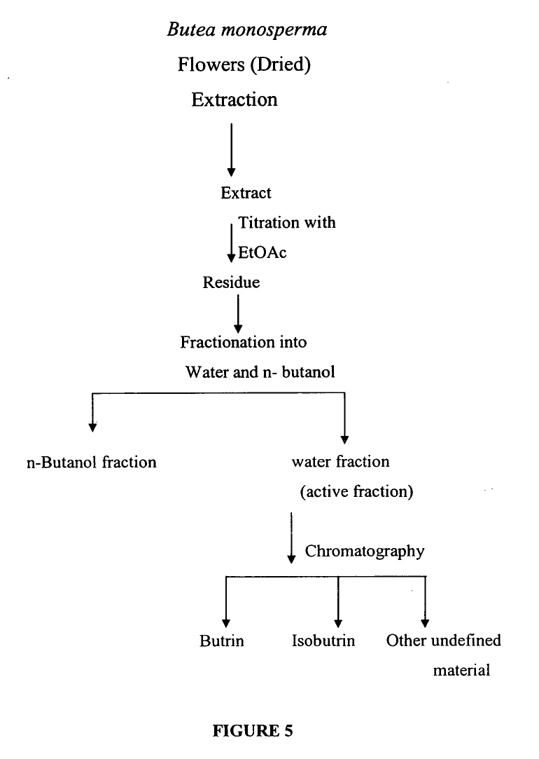

In an embodiment of the present invention, the organic phase used for partitioning the residue is n-butanol. A flowchart for isolation of active fraction from Butea monosperma is shown in FIG. 5.

The following examples are given by way of illustration of the present invention and should not be construed to limit the scope of present invention.

### EXAMPLE 1

500 gm of dried powdered flowers of Butea monosperma were soaked in 3 L distilled water and heated on steam bath for 4 hr. The aqueous extract was filtered through celite and concentrated on rotavapour at 50° C. to 250 ml. The extraction process was repeated thrice more and the combined concentrated aqueous extract (1 L) was freeze dried to give dry powder (145 g). This extract was triturated with ethyl acetate and the residue was taken in water (750 ml) and) and extracted with n-butanol (4×200 ml). The aqueous fraction was freeze dried to get active fraction (88 g)

### EXAMPLE 2

The shade dried, powdered flowers of Butea monosperma (1 kg) were soaked in rectified spirit and kept overnight. The extract was drained and filtered through celite. The extraction process was repeated thrice more. The rectified spirit was evaporated under reduced pressure to obtain a dark brown mass, and this extract was titrated with ethyl acetate. The residue left was dissolved in water and extracted with n-butanol (3×400 ml). The aqueous fraction was freeze dried to get active fraction (156 g).

### EXAMPLE 3

The shade dried, powdered flowers of Butea monosperma (1 kg) were soaked in methanol and kept overnight. The extract was drained and filtered through celite. The extraction process was repeated thrice more. The methanol was evaporated under reduced pressure to obtain a dark brown mass, and this extract was triturated with ethyl acetate. The residue left was dissolved in water (1 L) and extracted with n-butanol (3×400 ml). The aqueous fraction was freeze dried to yield dry powder (142 g).

### EXAMPLE 4

HPLC Analysis of Active Fraction:

The active fraction contains isobutrin and butrin minimum in the range of 2 to 4.5% and 9 to 12% by weight of the total extract.

Solvent system acetonitrile: 0.001M phosphoric acid (30:70), column RP18e (E. Merck, 5 um, 4.0×250 mm), column temperature 30°, flow rate 0.6 ml/min, wave length 254.

### EXAMPLE 5

Characterisation of Compounds 1 and 2:

Aqueous fraction (25 g) from the aqueous extract of Butea monosperma was chromatographed over a column of silica gel (600 g). Elution with ethyl acetate:methanol (85:15) gave 150 mg isobutrin (1) followed by 1.2 g butrin (2).

Isobutrin (1): m.p. 187-89°; M⁺ 596; ¹H NMR (200 Hz, DMSO-d6) showed signals at δ6.63 (2H, m, H-3′, H-5′), 6.90 (1H, d, J=8 Hz, H-5), 7.46 (1H,d, J=8 Hz, H-6), 7.72 (3H,m, H-2, H-α, H-β), 8.23 (1H,d, J=8 Hz, H-6′); IR (KBr) ν (cm⁻¹): 3386, 2981, 1633, 1572, 1518, 1421, 1363, 1284, 1219, 1124, 1072, 804

Butrin (2); m.p. 189-90°; M⁺ 596; ¹H NMR (200 MHz, DMSO-d6) showed signals at δ3.18 (2H, m, H-3), 5.45 (1H, dd, J=4, 12 Hz, H-2), 6.68 (1H, d, J=8 Hz, H-8), 6.72 (1H, d, J=8 Hz, H-6), 6.80 (1H,d, J=8 Hz, H-5′), 7.05 (1H,d, J=8 Hz, H-6′), 7.30 (1H,s, H-2′), 7.73 (1H,d, J=8 Hz, H-5) IR (KBr) ν (cm⁻¹): 3362, 2925, 1667, 1613, 1574, 1523, 1443, 1281, 1085, 860, 804.

### EXAMPLE 6

In Vitro Cytotoxicity of Aqueous Extract and Aqueous Fraction Against Human Cancer Cell Lines:

The human cancer cell lines procured from National Cancer Institute, Frederick, U.S.A or National Center for Cell Science; Pune, India. were used in present study. Cells were grown in tissue culture flasks in complete growth medium (RPMI-1640 medium with 2 mM glutamine, 100 μg/ml streptomycin, pH 7.4, sterilized by filtration and supplemented with 10% fetal calf serum and 100 units/ml penicillin before use) at 37° C. in an atmosphere of 5% CO₂ and 90% relative humidity in a carbon dioxide incubator. The cells at subconfluent stage were harvested from the flask by treatment with trypsin (0.5% in PBS containing 0.02% EDTA) for determination of cytotoxicity. Cells with viability of more than 98% as determined by trypan blue exclusion were used for assay. The cell suspension of the required cell density was prepared in complete growth medium with gentamycin (50 μg/ml) for determination of cytotoxicity.

A stock solutions of (20 mg/ml) of test material were prepared in distilled water. The stock solutions were serially diluted with complete growth medium containing 50 μg/ml of gentamycin to obtain working test solutions of required concentrations. In vitro cytotoxicity against human cancer cell lines was determined (Monks, A., Scudiero, D., Skehan, P., Shoemaker R., Paull, K., Vistica, D., Hose, C., Langley, j., Cronise, P., Vaigro-Wolff, A., Gray-Goodrich, M., Campbell, H., Mayo, J and Boyd, M. (1991). Feasibility of a high-flux anticancer drug screen using a diverse panel of cultured human tumor cell lines. J. Natl. Cancer Inst. 83, 757-766.) using 96-well tissue culture plates. The 100 μl of cell suspension was added to each well of the 96-well tissue culture plate. The cells were incubated for 24 hours. Test materials in complete growth medium (100 μl) were added after 24 hours incubation to the wells containing cell suspension. The plates were further incubated for 48 hours (at 37° C. in an atmosphere of 5% and 90% relative humidity in a carbon dioxide incubator) after addition of test material and then the cell growth was stopped by gently layering trichloroacetic acid (TCA, 50 μl, 50%) on top of the medium in all the wells. The plates were incubated at 4° C. for one hour to fix the cells attached to the bottom of the wells. The liquid of all the wells was gently pipetted out and discarded. The plates were washed five times with distilled water to remove TCA, growth medium low molecular weight metabolites, serum proteins etc and air-dried. Cell growth was measured by staining with sulforhodamine B dye (P. Skehan, R. Storeng, D. Scudiero, A. Monks, J. McMohan, D. Vistica, J. T. Warren, H. Bokesch, S. Kenney, M. R. Boyd (1990) New colorimetric cytotoxic Assay for Anticancer—Drug Screening Journal of the National Cancer Institute 82, 1107-1112). The adsorbed dye was dissolved in Tris-Buffer (100 μl, 0.01M, pH 10.4) and plates were gently stirred for 5 minutes on a mechanical stirrer. The optical density (OD) was recorded on ELISA reader at 540 nm.

The cell growth was calculated by subtracting mean OD value of respective blank from the mean OD value of experimental set. Percent growth in presence of test material was calculated considering the growth in absence of any test material as 100% and in turn percent growth inhibition in presence of test material will be calculated.

In vitro cytotoxicity (percent growth inhibition) of aqueous extract and aqueous fraction of Butea monosperma flowers against human cancer cell lines are summarized in Table 1.

TABLE 1
In vitro cytotoxicity (percent growth inhibition) of Extract and Fraction of Butea
monosperma flowers against human cancer cell lines.

| | Test material | | | | | | |
|---|---|---|---|---|---|---|---|
| | Concentration | Extract | Fraction | Adriamycin | Mitomycin C | Tamoxifen | 5-Flurouracil |
| Tissue | Cell line | 100 μg/ml | 100 μg/ml | 1 × 10⁻⁵ M | 1 × 10⁻⁵ M | 1 × 10⁻⁵ M | 2 × 10⁻⁵ M |
| Breast | MCF-7 | 6 | — | 72 | — | — | — |
| Breast | T 47 D | 4 | 0 | 34 | — | — | — |
| Breast | ZR 75-1 | 0 | — | 46 | — | — | — |
| Cervix | HeLa | 9 | — | — | — | — | — |
| Cervix | SiHa | 0 | 0 | — | — | — | — |
| CNS | IMR 32 | 81 | — | 87 | 83 | — | — |
| CNS | SK N MC | 23 | 2 | — | — | 27 | — |
| CNS | SK N SH | 43 | — | 82 | — | — | — |
| CNS | SNB 78 | 2 | — | 20 | — | — | — |
| Colon | Colo 205 | 87 | 0 | — | — | — | — |
| Colon | SW 620 | 95 | — | 59 | — | — | — |
| Colon | HCT 15 | — | 0 | — | — | — | 50 |
| Colon | HT 29 | — | 0 | 69 | — | — | — |
| Liver | Hep-2 | 51 | 35 | — | — | — | — |
| Lung | A 549 | 19 | 11 | — | | 17 | — |
| Lung | NCI-H23 | 0 | — | — | 59 | — | — |
| Oral | KB | 16 | — | — | — | — | 9 |
| Ovary | NIH | 0 | — | — | 31 | — | — |
| | OVCAR 3 | | | | | | |
| Ovary | NIH | — | 27 | 45 | — | 20 | — |
| | OVCAR5 | | | | | | |
| Ovary | OVCAR 5 | 5 | 6 | — | — | — | — |
| Prostate | DU 145 | 0 | — | — | 69 | — | — |

The aqueous extract of Butea monosperma flowers was evaluated for its in vitro cytotoxicity against number of human cancer cell lines namely breast (MCF-7, T-47-D and ZR-75-1), cervix (HeLa and SiHa), CNS (IMR-32, SK-N-MC, SK-N-SH and SNB-78), colon (Colo-205 and SW-620), liver (Hep-2), lung (A-549 and NCI-H23), oral (KB), ovary (NIH-OVCAR-3 and OVCAR-5) and prostate (DU-145) at a concentration of 100 μg/ml. It showed high degree of growth inhibition i.e. 95, 87 and 81% against SW-620, Colo-205 and IMR-32 human cancer cell lines respectively. The Hep-2, SK-N-SH and SK-N-MC human cancer cell lines showed moderate effect of 51, 43 and 23% respectively. The response towards A-549 (19%) and KB (16%) human cancer cell lines was of low degree. Rest of the human cancer cell lines showed poor or no response.

The aqueous fraction of Butea monosperma flowers was also evaluated for its in vitro cytotoxicity against number of human cancer cell lines namely breast (T-47-D), cervix (SiHa), CNS (SK-N-MC), colon (Colo-205, HCT-15 and HT-29), liver (Hep-2), lung (A-549), oral (KB) and ovary (NIH-OVCAR-5 and OVCAR-5) at a concentration of 100 μg/ml. It showed maximum growth inhibition of against Hep-2 (35%) followed by NIH-OVCAR-5 (27%) and A-549 (11%). Rest of the human cancer cell lines showed still less or no response.

### EXAMPLE 7

In Vivo Anticancer Activity of Aqueous Extract and Aqueous Fraction.

Transgenic mice: Development of the X-myc transgenic mice is described elsewhere (Kumar, V., Singh, M., Totey, S. M. and Anand, R. K. (2001). Bicistronic DNA construct comprising X-myc transgene for use in production of transgenic animal model systems for human hepatocellular carcinoma and transgenic animal model systems so produced. U.S. Pat. No. 6,274,788 B1). The animals were bred and cared as per guidelines of the CPCSEA (Project No. VIR-2, ICGEB, 2001). The transgene positive animals were selected at 4 weeks of age by the genomic tail DNA analysis using PCR (Kumar et al. 2001).

Drug treatment: Each animal received biweekly nine intra-peritoneal injections of either saline (control group) or saline containing drug (500 mg/Kg) (treatment group).

### EXAMPLE 8

Histopathological Studies and Other Parameters:

Animals of both control and treatment groups were sacrificed at 12 or 20 weeks of age and the gross appearances of liver were recorded. For histopathological examination, the samples were collected in 10% buffered-formalin and paraffin blocks were prepared. The morphological and cytological details of liver were investigated by light microscopy of the tissue sections (2-5 mm thick) stained with hematoxylin and eosin.

The level of VEGF in the sera of control and treated mice was measured using a mouse-specific ELISA kit (Oncogene Research Products, USA, Cat # QIA52). All the manipulations were done as per instruction of the supplier. The VEGF concentration was expressed as picogram/ml serum;

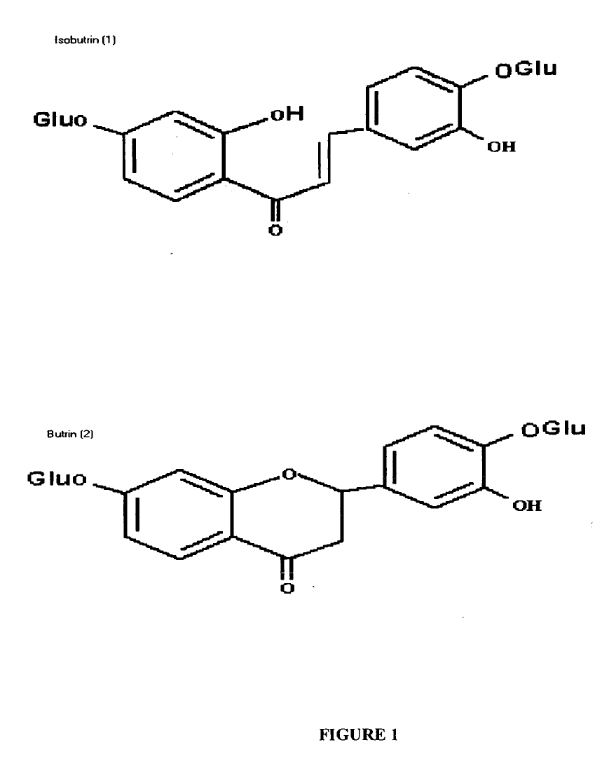

Results of histological studies and serum VEGF levels are shown in FIGS. 1-3 and Table 2 respectively.

TABLE 2
Serum VEGF levels (pg/ml) in X-myc mice after treatment of Butea
monosperma flowers extract.

Treatment

| | Control Group | Treated with Extract |
|---|---|---|
| Period | (n = 6) | (n = 6) |
| 12 weeks | 239.6 ± 31.4 | 76.1 ± 12.9* |
| 20 weeks | 237.3 ± 36.3 | 136.5 ± 16.7** |

Level in normal adult mice = 93.7 ± 10.8 pg/ml
Level of significance = *p < 0.001; **p < 0.01

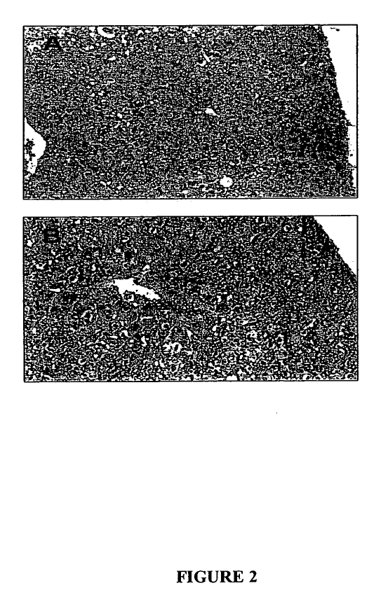

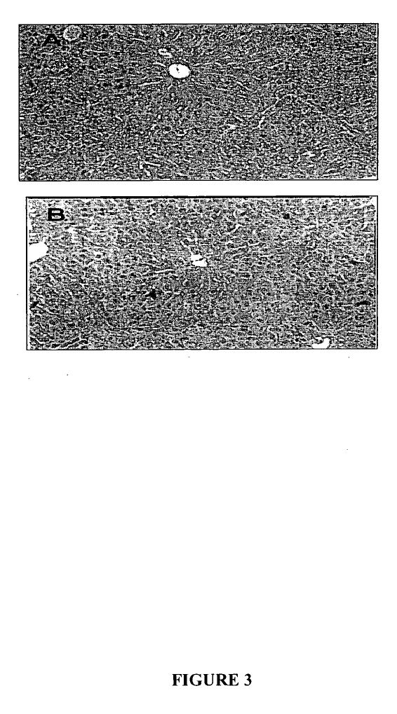

The liver of control animals (FIG. 1) showed atypical mitosis, dysplasia and loss of normal hepatic architecture. The malignant hepatocyte cords showed large pleiomorphic nuclei with multinucleation and macronucleoli. FIGS. 2 and 3 respectively show the effect of treatment with aqueous extract (A003) and aqueous fraction (F009) of the flower of Butea monosperma where the liver appeared to be normal both at 12 and 20 weeks post-treatment. The anticancer activity of Butea monosperma appears to relate to an anti-angiogenic function since the serum VEGF levels of treated animals (Table 2) was significantly down-regulated (p<0.001 to 0.01)″.

### EXAMPLE 9

In Vitro Cytotoxicity of Compounds Isolated from Aqueous Fraction Against Human Cancer Cell Lines:

Methodology is the same as given in example 6 except for stock solutions of 1×10⁻²M was prepared instead of 20 mg/ml.

The compounds were evaluated for its in vitro cytotoxicity against number of human cancer cell lines namely cervix (SiHa), CNS (SK-N-SH), colon (HT-29 HCT-15, Colo-205 and SW-620), lung (HOP-62) at a concentration of 1×10⁻⁴, 1×10⁻⁵ and 1×10⁻⁶ M. Both the compounds showed high degree of growth inhibition i.e. 40-99% at 1×10⁻⁴ M against the cell lines used. The maximum growth inhibition at 1×10⁻⁵ M was 26%. The compounds were inactive at 1×10^{31 6} M.

In vitro cytotoxicity (percent growth inhibition) of the compounds is summarized in Table 3

TABLE 3
In vitro cytotoxicity (percent growth inhibition) of Compounds (butrin and
isobutrin) against human cancer cell lines

| | | Human cancer cell lines | | | | | | |
|---|---|---|---|---|---|---|---|---|
| | | | | | SK- | | | Colo- |
| Compound | Conc. | HT-29 | SW620 | HCT-15 | NSH | HOP-62 | SiHa | 205 |
| Butrin | 1 × 10⁻⁶ M | 9 | 0 | 0 | 9 | 0 | 0 | 0 |
| Butrin | 1 × 10⁻⁵ M | 11 | 0 | 26 | 8 | 6 | 0 | 0 |
| Butrin | 1 × 10⁻⁴ M | 99 | 92 | 98 | 83 | 65 | 80 | 40 |
| Isobutrin | 1 × 10⁻⁶ M | 0 | 0 | 0 | 0 | 0 | 0 | 0 |
| Isobutrin | 1 × 10⁻⁵ M | 10 | 0 | 21 | 25 | 0 | 0 | 0 |
| Isobutrin | 1 × 10⁻⁴ M | 94 | 85 | 99 | 93 | 65 | 50 | 74 |

Advantages

The main advantages of the present invention are:
1. The invention relates to isolation of a novel extract/fraction having anticancer activity against hepatocellular carcinoma.
2. The present process utilizes highly economical raw material which is abundant in nature.
3. The concept used in the process makes it ideal and most easy to step up.

We claim:

1. A pharmaceutical composition useful for the treatment of hepatocellular carcinoma wherein the said composition comprising the therapeutically effective amount of an extract and/or its active fraction obtained from any plant parts of Butea monosperma or therapeutically effective amount of compound butrin and/or isobutrin or its derivatives or analogues or pharmaceutically acceptable salt thereof optionally along with one or more pharmaceutically acceptable carriers.

2. A pharmaceutical composition as claimed in claim 1, wherein the said composition comprising the therapeutically effective amount of an extract and/or its active fraction obtained from any plant parts of Butea monosperma optionally along with one or more pharmaceutically acceptable carriers.

3. A pharmaceutical composition as claimed in claim 2, wherein the dosage of the said composition is administered at a unit dose of at least 0.5 g/kg body weight.

4. A pharmaceutical composition as claimed in claim 1, wherein the said composition comprising the therapeutically effective amount of compound butrin and/or iso butrin or its derivatives or analogues or pharmaceutically acceptable salt thereof optionally along with one or more pharmaceutically acceptable carriers.

5. A pharmaceutical composition as claimed in claim 4, wherein the dosage of the said composition is administered at a unit dose of less than 0.5 g/kg body weight.

6. A pharmaceutical composition as claimed in claim 1, wherein the dosage of the said composition is administered in soluble form preferably in suspension form.

7. A pharmaceutical composition as claimed in claim 1, wherein the carrier used is selected from the group consisting of: saline, gum acacia and carboxy methyl cellulose.

8. A pharmaceutical composition as claimed in claim 1, wherein the administration route is selected from the group consisting of intraperitoneal, intravenous, intramuscular and oral.

9. A method of treating hepatocellular carcinoma in a subject, wherein the said method comprising the step of administering to the subject a pharmaceutical composition comprising the therapeutically effective amount of an extract and/or its active fraction obtained from any plant parts of Butea monosperma or therapeutically effective amount of compound butrin and/or isobutrin or its derivatives or analogues or pharmaceutically acceptable salt thereof optionally along with one or more pharmaceutically acceptable carriers.

10. A method as claimed in claim 9, wherein the subject used is selected from the group consisting of humans and mammals.

11. A method as claimed in claim 9, wherein the said method comprising the step of administering to the subject a pharmaceutical composition as claimed in claim 2.

12. A method as claimed in claim 11, wherein the dosage of the said composition administered is at a unit dose of at least 0.5 g/kg body weight.

13. A method as claimed in claim 9, wherein the said method comprising the step of administering to the subject a pharmaceutical composition as claimed in claim 4.

14. A method as claimed in claim 13, wherein the dosage of the said formulation administered is at a unit dose of less than 0.5 g/kg body weight.

15. A method as claimed in claim 9, wherein the dosage of the said composition is administered in soluble form preferably in suspension form.

16. A method as claimed in claim 9, wherein the carrier used is selected from the group comprising of saline, gum acacia, carboxy methyl cellulose or any other known pharmaceutically acceptable carrier.

17. A method as claimed in claim 9, wherein the administration route is selected from the group consisting of intraperitoneal, intravenous, intramuscular and oral.

18. Use of the extract and bioactive fraction obtained from Butea monosperma in the treatment of hepatocellular carcinoma.

19. Use of the compound butrin and isobutrin in the treatment of the hepatocellular carcinoma.

20. A process for isolating the bioactive fraction comprising of butrin and/or isobutrin from any plant parts of Butea monosperma, wherein the said process comprising:
a) powdering the plant material;
b) extracting the powder obtained from step (a) by percolation using solvents selected from the group comprising of ethanol, methanol, water, individually or in combination thereof to obtain extract;
c) concentrating the extract obtained from step (b) under reduced pressure at <50° C.;
d) titrating the extract obtained from step (c) with solvents selected from the group comprising of ethylene chloride, methylene chloride, chloroform &/or ethyl acetate to get residue;
e) partitioning the residue obtained from step (d) between aqueous phase and organic phase;
f) drying the aqueous part obtained from step (e) to get desired active fraction by known methods.

21. A process as claimed in claim 21, wherein the organic phase used for partitioning the residue is n-butanol.
